(19) United States
(12) Patent Application Publication
(10) Pub. No.: US 2022/0229002 A1
(43) Pub. Date: Jul. 21, 2022

(54) TIRE ELECTRICAL RESISTANCE MEASUREMENT DEVICE AND ELECTRICAL RESISTANCE PROBE

(71) Applicant: MITSUBISHI HEAVY INDUSTRIES MACHINERY SYSTEMS, LTD., Hyogo (JP)

(72) Inventors: Jiro AGAWA, Kobe-shi (JP); Takumi TSUMURA, Kobe-shi (JP); Yoshikazu NISHIHARA, Kobe-shi (JP); Tatsuya UEDA, Kobe-shi (JP)

(21) Appl. No.: 17/611,230

(22) PCT Filed: May 20, 2019
(86) PCT No.: PCT/JP2019/019890

§ 371 Date: Nov. 15, 2021

(51) Int. Cl. G01N 27/04; G01M 17/02

(52) U.S. Cl. CPC G01N 27/041; G01M 17/02

(57) ABSTRACT

This tire electrical resistance measurement device is provided with an inner circumferential-side probe and an outer circumferential-side probe. The inner circumferential-side probe is disposed on the inner circumferential side of a tire and is capable of coming into contact with the inner circumference of the tire. The outer circumferential-side probe is disposed on the outer circumferential side of the tire and is capable of coming into contact with a tread portion of the tire by moving relative to the tire in a radial direction of the tire. The outer circumferential-side probe extends in the width direction of the tire and is deformable in the radial direction so as to follow a protrusion-recess shape of the tread portion in the width direction. The outer circumferential-side probe is electrically conductive at least at a contact surface that comes into contact with the tread portion.

### TECHNICAL FIELD

The present invention relates to a tire electric resistance measurement device and an electric resistance probe.

### BACKGROUND ART

In general, a vehicle, such as an automobile, is designed such that, in a case where a body is charged, electric charge escapes into the ground through a tire.

Accordingly, to secure that electric charge can stably escape into the ground, in a period from when a step, such as vulcanization molding of the tire, ends until shipment, there is a case where an inspection step of inspecting an electric resistance between an inner peripheral portion and a tread part of the tire is performed. In inspecting the electric resistance of the tire, an inner side probe is brought into contact with the inner peripheral portion of the tire, and an outer side probe is brought into contact with the tread part.

For example, PTL 1 discloses a configuration in which an outer side probe capable of being brought into contact with a tread part of a tire is curvedly deformable along the shape of the tire from a central portion to a shoulder portion of the tread part in a width direction of the tire. In this configuration, the outer side probe is made of a linear electric conductor that stretches between an end portion of a longitudinal frame and an end portion of a transverse frame. In an outer peripheral surface of the tire, a low electric resistance portion made of a material having low electric resistance is exposed in a part of the tire in the width direction. In PTL 1, the outer side probe made of the linear electric conductor is brought into contact with the outer peripheral surface of the tire, whereby the outer side probe is brought into contact with the low electric resistance portion.

### CITATION LIST

### Patent Literature

[PTL 1] Japanese Patent No. 5943810

### SUMMARY OF INVENTION

### Technical Problem

In an inspection step of measuring electric resistance as in PTL 1 described above, the tire is often inspected in a single tire state in which the tire is not mounted on a wheel and is not filled with air. In a case of inspecting the single tire in this way, a part of the tread part of the tire may be dented to the inside in a radial direction of the tire and a dent may be formed. However, the electric conductor of the outer side probe disclosed in PTL 1 cannot enter the dent. For this reason, in a case where the low electric resistance portion is disposed in the dent of the tread part of the tire, there is a possibility that the electric conductor of the outer side probe is not brought into contact with the low electric resistance portion, and the electric resistance of the tire cannot be correctly measured.

An object of the invention is to provide a tire electric resistance measurement device and an electric resistance probe capable of improving reliability in electric resistance measurement of a tire.

### Solution to Problem

According to a first aspect of the invention, there is provided a tire electric resistance measurement device including an inner side probe and an outer side probe. The inner side probe is disposed on an inner periphery side of a tire and is capable of being brought into contact with an inner peripheral portion of the tire. The outer side probe is disposed on an outer periphery side of the tire and is capable of being brought into contact with a tread part of the tire by relatively moving in a radial direction of the tire with respect to the tire. The outer side probe extends in a width direction of the tire and is deformable following the radial direction corresponding to an undulating shape of the tread part in the width direction. The outer side probe has electric conductivity in at least a contact surface of the deformable portion with the tread part.

According to such a configuration, the outer side probe is deformable following the radial direction corresponding to the undulating shape of the tread part in the width direction of the tire. With this, in a case where the outer peripheral surface is dented to an inside in the radial direction of the tire in a part of the tire in the width direction, the outer side probe enters the portion dented to the inside in the radial direction. Then, the contact surface of the outer side probe having electric conductivity is brought into contact with the outer peripheral surface of the tire even in the portion dented to the inside in the radial direction of the tire. Therefore, even in a case where a low electric resistance portion is positioned in the portion dented to the inside in the radial direction of the tire, the outer side probe is brought into contact with the low electric resistance portion, whereby it is possible to improve reliability in electric resistance measurement of the tire.

According to a second aspect of the invention, the tire electric resistance measurement device may be configured such that the outer side probe of the first aspect enters a dent more dented to an inside in the radial direction than a maximum outer diameter portion of the tire in an intermediate portion of the tire in the width direction in a case where the outer side probe is brought into contact with the tread part of the tire by relatively moving in the radial direction of the tire with respect to the tire.

With this, the deformable portion enters the dent more dented to the inside in the radial direction than the maximum outer diameter portion of the tire in the intermediate portion of the tire in the width direction. Therefore, even in a case where a low electric resistance portion is positioned in the portion dented to the inside in the radial direction of the tire, it is possible to bring the outer side probe into contact with the low electric resistance portion.

According to a third aspect of the invention, the tire electric resistance measurement device may further include a support member that has rigidity higher than the outer side probe of the first aspect, extends in the width direction outside the tire in the radial direction with respect to the outer side probe, and supports the outer side probe.

With this, when the outer side probe is brought into contact with the tread part of the tire and is deformed in the radial direction corresponding to the undulating shape of the tread part in the width direction, the support member firmly supports the outer side probe on an outside in the radial direction. With this, it is possible to make the outer side probe enter the dent more dented to the inside in the radial direction than the maximum outer diameter portion of the tire.

According to a fourth aspect of the invention, the tire electric resistance measurement device may be configured such that the outer side probe of the first aspect includes a driven displaceable portion and a pressing portion. The driven displaceable portion is displaced to an outside in the radial direction corresponding to the undulating shape of the tread part of the tire in a case where the driven displaceable portion is brought into contact with the tread part of the tire by relatively moving in the radial direction of the tire with respect to the tire. The pressing portion presses the driven displaceable portion to an inside in the radial direction of the tire.

With this, in a case where the driven displaceable portion is brought into contact with the tread part of the tire by relatively moving in the radial direction with respect to the tire, the driven displaceable portion is displaced to be press-fitted to the outside in the radial direction corresponding to the undulating shape of the tread part of the tire. Since the driven displaceable portion is pressed to the inside in the radial direction of the tire by the pressing portion, the driven displaceable portion enters the dent more dented to the inside in the radial direction than the maximum outer diameter portion of the tire. Therefore, even in a case where a low electric resistance portion is positioned in the portion dented to the inside in the radial direction of the tire, it is possible to bring the outer side probe into contact with the low electric resistance portion.

According to a fifth aspect of the invention, the tire electric resistance measurement device may be configured such that the driven displaceable portion of the fourth aspect is a band-shaped member that extends in the width direction and has flexibility and electric conductivity.

With this, the driven displaceable portion made of the band-shaped member that extends in the width direction of the tire and has flexibility and electric conductivity enters the dent more dented to the inside in the radial direction than the maximum outer diameter portion of the tire. Therefore, even in a case where a low electric resistance portion is positioned in the portion dented to the inside in the radial direction of the tire, it is possible to bring the outer side probe into contact with the low electric resistance portion.

According to a sixth aspect of the invention, the tire electric resistance measurement device may be configured such that the driven displaceable portion of the fourth aspect is a plurality of advance/retreat members that are provided at intervals in the width direction and are provided advanceable and retreatable in the radial direction.

With this, each of the advance/retreat members configuring the driven displaceable portion is displaced to be press-fitted to the outside in the radial direction corresponding to the undulating shape of the tread part of the tire in a case where each of the advance/retreat members is brought into contact with the tread part of the tire by relatively moving in the radial direction with respect to the tire. Since a plurality of advance/retreat members are pressed to the inside in the radial direction of the tire by the pressing portion, a plurality of advance/retreat members enter the dent more dented to the inside in the radial direction than the maximum outer diameter portion of the tire. Therefore, even in a case where a low electric resistance portion is positioned in the portion dented to the inside in the radial direction of the tire, it is possible to bring the outer side probe into contact with the low electric resistance portion.

According to a seventh aspect of the invention, the tire electric resistance measurement device may be configured such that the pressing portion of the fourth aspect is formed to be compressible by being elastically deformed toward the outside in the radial direction corresponding to the undulating shape of the tread part of the tire in a case where the pressing portion is brought into contact with the tread part of the tire by relatively moving in the radial direction of the tire with respect to the tire.

With this, since the pressing portion is elastically deformed toward the outside in the radial direction and compressed, and exerts pressing force toward the inside in the radial direction, the driven displaceable portion that is displaced to be press-fitted to the outside in the radial direction corresponding to the undulating shape of the tread part of the tire is pressed to the inside in the radial direction of the tire by the pressing force of the pressing portion. With this, it is possible to make the driven displaceable portion enter the dent more dented to the inside in the radial direction than the maximum outer diameter portion of the tire.

According to an eighth aspect of the invention, the outer side probe of the first aspect is elastically deformable toward an outside in the radial direction corresponding to the undulating shape of the tread part of the tire in a case where the outer side probe is brought into contact with the tread part of the tire by relatively moving in the radial direction of the tire with respect to the tire, and has electric conductivity.

With this, since the outer side probe is elastically deformable and has electric conductivity, in a case where a part of the tire in the width direction is dented to the inside in the radial direction of the tire, the outer side probe enters the portion dented to the inside in the radial direction. Then, the outer side probe is brought into contact with the outer peripheral surface of the tire over the entire tire in the width direction. Therefore, even in a case where a low electric resistance portion is positioned in the portion dented to the inside in the radial direction of the tire, it is possible to bring the outer side probe into contact with the low electric resistance portion to inspect the electric resistance of the tire. Furthermore, since the outer side probe has electric conductivity, it is possible to efficiently perform manufacturing or the like of the outer side probe compared to a case where only a contact surface has electric conductivity.

According to a ninth aspect of the invention, there is provided an electric resistance probe that extends in a width direction of a tire, is deformable following a radial direction of the tire corresponding to an undulating shape of the tire in the width direction in a case where the electric resistance probe is brought into contact with the tire by relatively moving in the radial direction of the tire with respect to the tire, and has electric conductivity in at least a contact surface with the tire.

In a case where such an electric resistance probe is applied to at least one of the outer side probe and the inner side probe of the tire electric resistance measurement device of any one of the first to eighth aspects, when the electric resistance probe is brought into contact with the tire, it is possible to deform the electric resistance probe following the radial direction of the tire corresponding to the undulating shape of the tire. For this reason, even though there is an undulating shape, for example, it is possible to bring the electric resistance probe into contact with a low electric resistance portion exposed in a tread part or an electric conduction portion exposed in a bead portion. Therefore, it is possible to improve reliability in electric resistance measurement of a tire.

### Advantageous Effects of Invention

With the tire electric resistance measurement device and the electric resistance probe described above, it is possible to improve reliability in electric resistance measurement of a tire.

### BRIEF DESCRIPTION OF DRAWINGS

FIG. 1 is a configuration diagram showing the schematic configuration of an electric resistance measurement device in a first embodiment of the invention.

FIG. 2 is a partial sectional view showing a main part of the electric resistance measurement device.

FIG. 3 is a plan view showing disposition of an outer side probe and an inner side probe of the electric resistance measurement device.

FIG. 4 is a side view showing the outer side probe of the electric resistance measurement device.

FIG. 5 is a diagram showing the outer side probe of the electric resistance measurement device and is a sectional view taken along an arrow A-A of FIG. 4.

FIG. 6 is a sectional view showing a state in which the outer side probe of the electric resistance measurement device is pressed to an outer peripheral surface of a tire.

FIG. 7 is a sectional view showing a state in which an outer side probe of an electric resistance measurement device in a modification example of the first embodiment of the invention is pressed to a tread part of the tire.

FIG. 8 is a sectional view showing a state in which an outer side probe of an electric resistance measurement device in a second embodiment of the invention is pressed to a tread part of a tire.

FIG. 9 is a sectional view showing a state in which an outer side probe of an electric resistance measurement device in a third embodiment of the invention is pressed to a tread part of a tire.

FIG. 10 is a diagram showing an inner side probe in a modification example of an embodiment of the invention.

### DESCRIPTION OF EMBODIMENTS

### First Embodiment

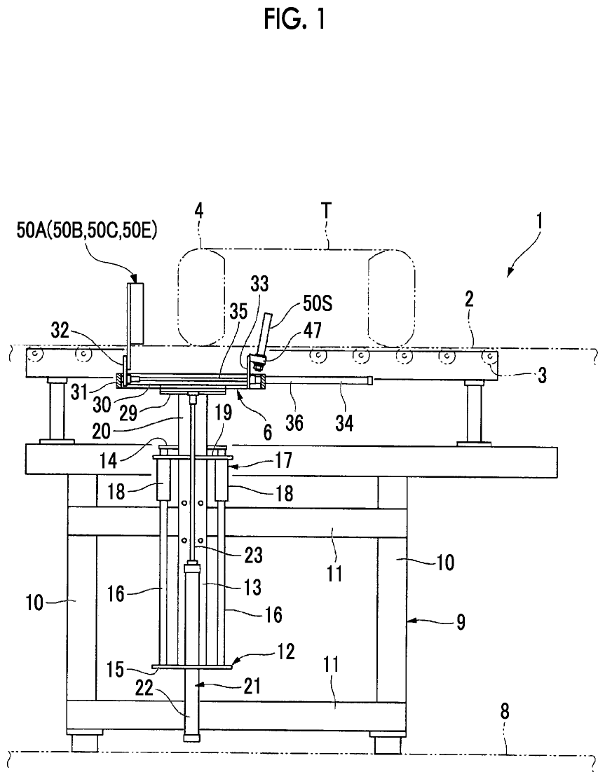

FIG. 1 is a configuration diagram showing the schematic configuration of an electric resistance measurement device in a first embodiment of the invention.

As shown in FIG. 1, an electric resistance measurement device 1 in the first embodiment is disposed on an inspection line (not shown) of a vulcanized tire T. The electric resistance measurement device 1 includes a roller conveyor 2 and a probe unit 6.

The roller conveyor 2 transfers the tire T. The roller conveyor 2 includes a plurality of rotatable rollers 3 that are arranged in a transfer direction. A plurality of rollers 3 are separated on both sides in a width direction of the roller conveyor 2 (hereinafter, simply referred to as a width direction). The roller conveyor 2 transfers the tire T in a state in which side walls 4 are turned in an up-down direction.

In FIG. 1, the rollers 3 at positions overlapping a probe unit 6 as viewed from the front are not shown.

The roller conveyor 2 is provided on a stand 9. The stand 9 is provided erect on the floor 8. The stand 9 includes a plurality of leg portions 10, beams 11, and a lifting/lowering mechanism 12.

A plurality of leg portions 10 extend in the up-down direction. The beams 11 are provided in upper portions and lower portions of the leg portions 10. The beams 11 extend in a horizontal direction and are attached to stretch between adjacent leg portions 10.

The lifting/lowering mechanism 12 lifts and lowers the probe unit 6. In the embodiment, a case where the lifting/lowering mechanism 12 is attached to the upper beam is illustrated. The lifting/lowering mechanism 12 includes a base portion 13, an upper support plate 14, a lower support plate 15, guide rods 16, a guide portion 17, a support arm 20, and a fluid pressure cylinder 21.

The base portion 13 extends in the up-down direction. The base portion 13 is fixed to the beam 11 slightly above a central portion in the up-down direction through a bracket (not shown).

The upper support plate 14 is provided at an upper end of the base portion 13. The upper support plate 14 extends in the horizontal direction.

The lower support plate 15 is provided at a lower end of the base portion 13. The lower support plate 15 faces the upper support plate 14.

The guide rods 16 are provided between the upper support plate 14 and the lower support plate 15. Two guide rods 16 are provided. The guide rods 16 extend in the up-down direction and are provided in parallel with each other. The guide rods 16 are disposed on both the outer sides of the base portion 13 in the width direction.

The guide portion 17 is liftably attached to the guide rods 16. The guide portion 17 includes two guide tubes 18 and a frame portion 19. The guide rods 16 are inserted into the two guide tubes 18, respectively. The frame portion 19 connects the upper end portions of the guide tubes 18.

The support arm 20 is formed in the frame portion 19 and extends upward. An upper end of the support arm 20 is fixed to a lower surface of the probe unit 6.

The fluid pressure cylinder 21 is a driving source that lifts and lowers the probe unit 6. The fluid pressure cylinder 21 includes an outer tube 22 and an inner rod 23. The outer tube 22 extends in the up-down direction and is fixed to the lower support plate 15. The inner rod 23 extends upward of the outer tube 22. An upper end of the inner rod 23 is fixed to the lower surface of the probe unit 6.

Such a fluid pressure cylinder 21 advances and retreats the inner rod 23 in the up-down direction due to differential pressure caused by supplying and discharging a compressed fluid into a cylinder chamber (not shown) of the outer tube 22. That is, the inner rod 23 of the fluid pressure cylinder 21 is displaced in a contraction direction, whereby the probe unit 6 moves downward along the guide rods 16 through the guide portion 17. With this, the probe unit 6 is moved in a downward direction being separated from the roller conveyor 2. The inner rod 23 of the fluid pressure cylinder 21 is displaced in an expansion direction, whereby the probe unit 6 moves upward along the guide rods 16 through the guide portion 17. With this, the probe unit 6 is moved upward, that is, a direction approaching the roller conveyor 2.

The probe unit 6 measures the electric resistance of the tire T. The probe unit 6 includes a base plate 29, a frame body 31, a guide rod 30, a first slide portion 32, a second slide portion 33, a fluid pressure cylinder 34 for a probe, outer side probes (electric resistance probes) 50A, and an inner side probe 50S.

The base plate 29 is fixed to an upper end portion of the inner rod 23. The frame body 31 is attached to the base plate 29. The frame body 31 supports the guide rod 30. The guide rod 30 extends in the transfer direction in the roller conveyor 2. The first slide portion 32 and the second slide portion 33 are slidably attached to the guide rod 30.

The fluid pressure cylinder 34 for a probe is a driving source that relatively moves the first slide portion 32 and the second slide portion 33. The fluid pressure cylinder 34 for a probe is attached to the first slide portion 32 and the second slide portion 33. The fluid pressure cylinder 34 for a probe includes an outer tube 36 and an inner rod 35. The inner rod 35 is provided retractably with respect to the outer tube 36. An end portion of the inner rod 35 is fixed to the first slide portion 32. The outer tube 36 is fixed to the second slide portion 33. In the embodiment, an end portion of the outer tube 36 on a side where the inner rod 35 protrudes is fixed to the second slide portion 33.

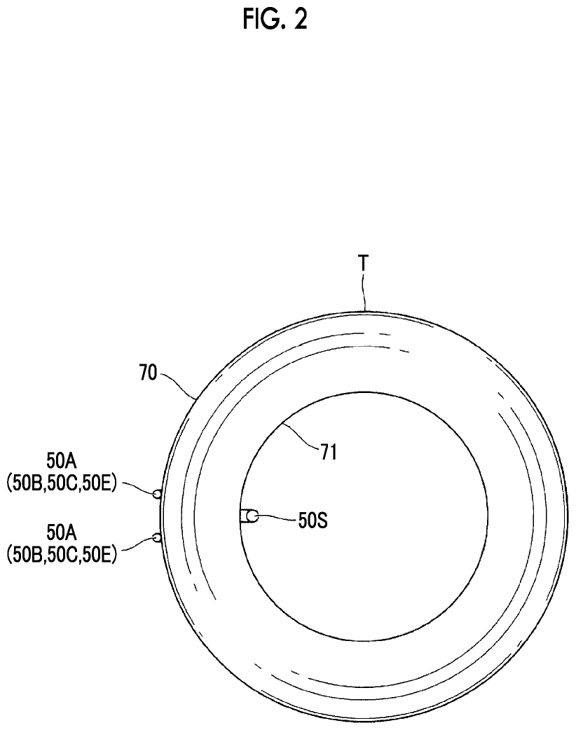

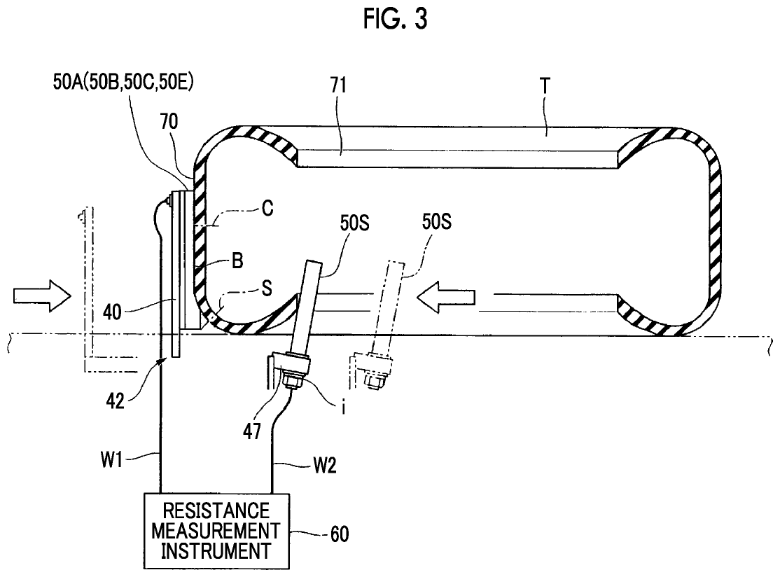

FIG. 2 is a partial sectional view showing a main part of the electric resistance measurement device. FIG. 3 is a plan view showing the disposition of the outer side probes and the inner side probe of the electric resistance measurement device.

As shown in FIG. 2, two outer side probes 50A are disposed in parallel at a predetermined interval in a circumferential direction of the tire T (hereinafter, simply referred to as a circumferential direction). In the following description, a “radial direction” means a radial direction of the tire T that is a tire to be measured.

As shown in FIG. 3, the outer side probe 50A is disposed on the outside (on outer periphery side) of a tread part (outer peripheral portion) 70 of the tire T in the radial direction at the time of electric resistance measurement of the tire T. The inner side probe 50S is disposed between the two outer side probes 50A and is disposed on the inside (on the inner periphery side) in the radial direction from the outer side probes 50A, in the circumferential direction. The inner side probe 50S is disposed on the inside (on the inner periphery side) in the radial direction from a bead portion (inner peripheral portion) 71 of the tire T at the time of electric resistance measurement of the tire T.

Each outer side probe 50A is fixed to the first slide portion 32 through a first support metal fitting 42. The outer side probe 50A is electrically insulated from the first support metal fitting 42 through an insulating member (not shown). The detailed configuration of the outer side probe 50A will be described below.

The inner side probe 50S is attached to the second slide portion 33 through a second support metal fitting 47. The second support metal fitting 47 extends inclined from an upper end portion of the second slide portion 33 toward a portion slightly below a side opposite to the first slide portion 32. The inner side probe 50S extends upward from an upper surface of the second support metal fitting 47. The inner side probe 50S in the embodiment extends in a direction perpendicular to the upper surface of the second support metal fitting 47. Similarly to the outer side probe 50A, the inner side probe 50S is also electrically insulated from the second support metal fitting 47 through an insulating member i.

The outer side probe 50A and the inner side probe 50S are driven to be lifted and lowered in the up-down direction by the drive of the fluid pressure cylinder 21. The outer side probe 50A and the inner side probe 50S can protrude upward from between the portions of the roller conveyor 2 separated from each other in the width direction at the time of electric resistance measurement of the tire T.

The outer side probe 50A and the inner side probe 50S can move in a direction approaching each other and in a direction being separated from each other by the drive of the fluid pressure cylinder 34 for a probe.

The outer side probe 50A relatively moves in the radial direction with respect to the tire T to be brought into contact with the tread part 70 formed in the outer peripheral portion of the tire T. The inner side probe 50S relatively moves in the radial direction with respect to the tire T to be brought into contact with the bead portion 71 formed in the inner peripheral portion of the tire T.

In the embodiment, the fluid pressure cylinder 34 for a probe is driven in a compression direction, whereby the first slide portion 32 and the second slide portion 33 are relatively displaced in a direction approaching each other along the guide rod 30. The outer side probe 50A and the inner side probe 50S are displaced in the direction approaching each other in this way, whereby it is possible to sandwich the tire T using the outer side probe 50A and the inner side probe 50S. On the other hand, in a case where the fluid pressure cylinder 34 for a probe is driven in an expansion direction, the first slide portion 32 and the second slide portion 33 are relatively displaced in a direction being separated from each other along the guide rod 30. The outer side probe 50A and the inner side probe 50S are displaced in a direction being separated from each other, whereby the outer side probe 50A and the inner side probe 50S are separated from the tire T.

The fluid pressure cylinder 34 for a probe shown in the embodiment is supported in a floating state in which the inner rod 35 and the outer tube 36 are displaceable together along the guide rod 30. For example, in a case where the fluid pressure cylinder 34 for a probe is driven in the compression direction, first, any one of the outer side probe 50A and the inner side probe 50S is brought into contact with the tire T and is stopped. Thereafter, in a case where the fluid pressure cylinder 34 for a probe is continuously driven in the compression direction, only the other one of the outer side probe 50A and the inner side probe 50S relatively moves in a direction approaching the tire T.

For example, in a case where the fluid pressure cylinder 34 for a probe is driven in the expansion direction, first, any one of the outer side probe 50A and the inner side probe 50S is brought into contact with the frame body 31 and is stopped. Thereafter, in a case where the fluid pressure cylinder 34 for a probe is continuously driven in the expansion direction, only the other one of the outer side probe 50A and the inner side probe 50S moves in a direction being separated from the tire T.

A support structure of the fluid pressure cylinder 34 for a probe is in the floating state in this way, whereby it is possible to appropriately sandwich the tire T using the outer side probe 50A and the inner side probe 50S even though a transfer position of the tire T is slightly shifted.

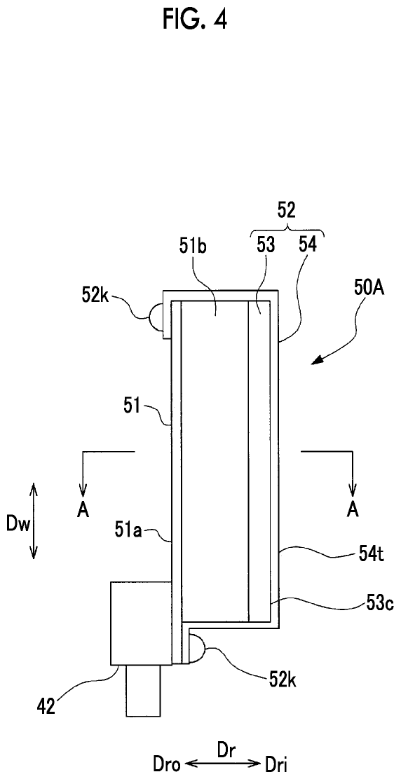

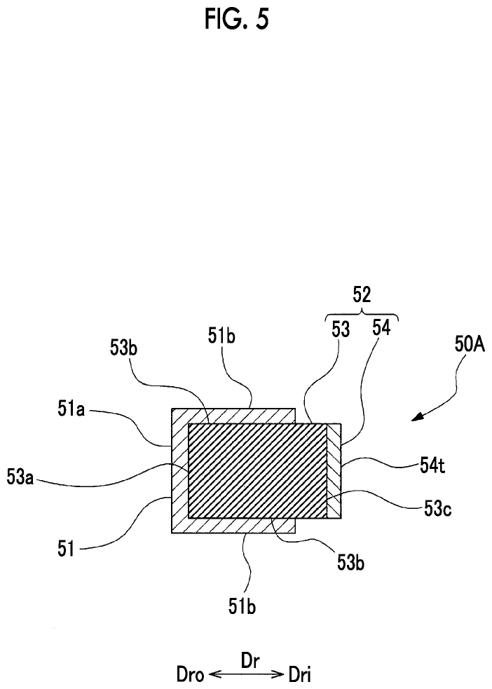

FIG. 4 is a side view showing the outer side probe of the electric resistance measurement device. FIG. 5 is a diagram showing the outer side probe of the electric resistance measurement device and is a sectional view taken along an arrow A-A of FIG. 4.

As shown in FIGS. 4 and 5, the outer side probe 50A comprises a support member 51 and a deformable portion 52. In the following description, the radial direction of the tire T is referred to as a “radial direction Dr”, the outside in the radial direction Dr is referred to as an “outside Dro”, and the inside in the radial direction Dr is referred to as an “inside Dri”. The width direction of the tire T is referred to as a “width direction Dw”.

The support member 51 is fixed to the first support metal fitting 42. Specifically, the support member 51 is fixed to the first support metal fitting 42 to extend in the width direction Dw of the tire T at the time of electric resistance measurement of the tire T. The support member 51 supports the deformable portion 52. The support member 51 has, for example, a base portion 51a and a pair of side wall portions 51b.

The base portion 51a is formed in a plate shape spreading in the circumferential direction and the width direction Dw of the tire T. A pair of side wall portions 51b extend from edge portions on both sides of the base portion 51a in the width direction Dw toward the inside Dri in the radial direction Dr of the tire T. The support member 51 includes the base portion 51a and a pair of side wall portions 51b, and thus, has a U-shaped section as viewed from the width direction Dw of the tire T. The support member 51 is made of, for example, metal, resin, or a fiber-strengthened material, and has rigidity higher than the deformable portion 52 described below.

The deformable portion 52 includes an elastically deformable body (pressing portion) 53 and a conductive portion (driven displaceable portion) 54.

As shown in FIG. 5, the elastically deformable body 53 is housed inside the support member 51 formed to have the U-shaped section. The elastically deformable body 53 has a base surface 53a that is directed toward the outside Dro in the radial direction Dr, two side surfaces 53b that extend from the base surface 53a to the inside Dri in the radial direction Dr, and a tip surface 53c that is directed toward the inside Dri in the radial direction Dr.

The base surface 53a is brought into contact with the base portion 51a. The two side surfaces 53b are brought into contact with a pair of side wall portions 51b, respectively. The tip surface 53c protrudes to the inside Dri in the radial direction Dr more than a pair of side wall portions 51b.

As shown in FIGS. 4 and 5, the elastically deformable body 53 extends in the width direction Dw of the tire T. The elastically deformable body 53 is deformable following the radial direction Dr corresponding to an undulating shape of the tread part 70 in the width direction Dw. The elastically deformable body 53 is formed of, for example, an easily elastically deformable material, such as rubber or sponge. Undulation due to grooves formed in the tread part 70 of the tire T is not included in the undulating shape.

The outer side probe 50A is relatively moved to the inside Dri in the radial direction Dr of the tire T with respect to the tire T, whereby the deformable portion 52 presses the tread part 70 of the tire T. In this case, the elastically deformable body 53 is compressed and deformed (elastically deformed) toward the outside Dro in the radial direction Dr corresponding to the undulating shape of the tread part 70 of the tire T.

The magnitude of the compression and deformation of the elastically deformable body 53 corresponds to the undulating shape of the tread part 70, and compressive deformation is greater in a protrusion than in a dent of the undulating shape. The compressed and deformed elastically deformable body 53 energizes the conductive portion 54 toward the inside Dri in the radial direction Dr of the tire T with elasticity.

The conductive portion 54 is attached to the tip surface 53c of the elastically deformable body 53. In other words, the conductive portion 54 is provided in a contact surface of the deformable portion 52 that is brought into contact with the tread part 70 of the tire T. The conductive portion 54 (band-shaped member 54t) has electric conductivity. The conductive portion 54 extends in the width direction Dw of the tire T. The conductive portion 54 has flexibility capable of following the deformation of the tip surface 53c of the elastically deformable body 53 corresponding to the undulating shape of the tread part 70. The conductive portion 54 shown in the embodiment is the band-shaped member 54t made of a commercially available conductive tape or the like. As the band-shaped member 54t, for example, a material having electric conductivity (in other words, having extremely low electric resistance), such as copper, silver, or aluminum.

As shown in FIG. 4, both end portions of the conductive portion 54 are fixed to the support member 51 by screws 52k or the like. In a case where the conductive portion 54 is brought into contact with the tread part 70 of the tire T by relatively moving in the radial direction Dr of the tire T with respect to the tire T, the conductive portion 54 is sandwiched between the tread part 70 and the tip surface 53c and is deformed following the deformation of the tip surface 53c of the elastically deformable body 53. That is, the conductive portion 54 is deformed along the undulating shape of the tread part 70 of the tire T.

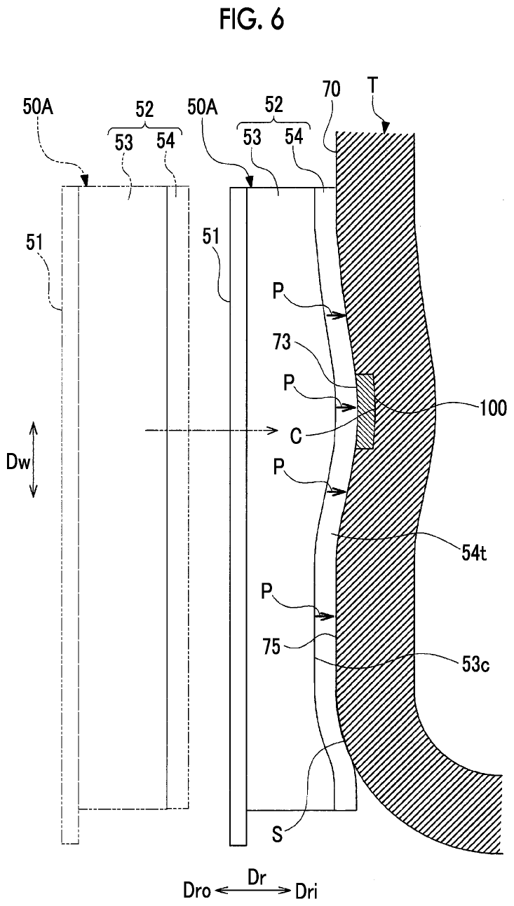

FIG. 6 is a sectional view showing a state in which the outer side probe of the electric resistance measurement device is pressed to the tread part of the tire.

As shown in FIG. 6, in a state in which the tire T that is filled with a fluid, such as air or nitrogen gas, for use is not filled with the fluid, there is a case where a part of the tread part 70 (outer peripheral portion) in the width direction Dw of the tire T is dented to the inside Dri in the radial direction Dr. In the embodiment, for example, a case where a dent 73 (dent) more dented to the inside Dri in the radial direction Dr than a maximum outer diameter portion 75 of the tire T in an intermediate portion in the width direction Dw of the tire T in the tread part 70 of the tire T.

With the above-described outer side probe 50A, the deformable portion 52 relatively moves in the radial direction Dr of the tire T with respect to the tire T and is pressed to the tread part 70 of the tire T. In this case, the outer side probe 50A is brought into contact in a range from a center portion C to a shoulder portion S of the tread part 70 in the width direction Dw of the tire T (in other words, the axial direction of the tire T).

More specifically, the elastically deformable body 53 and the conductive portion 54 of the deformable portion 52 are pressed to the tread part 70 to be deformed along the undulating shape of the tread part 70 of the tire T in the width direction Dw. In this case, the elastically deformable body 53 is compressed and deformed toward the outside Dro in the radial direction Dr corresponding to the undulating shape of the tread part 70 of the tire T. The compressed and deformed elastically deformable body 53 exerts pressing force P toward the inside Dri in the radial direction Dr and energizes the conductive portion 54 with elasticity. With this, the conductive portion 54 enters a dent 73 formed in the intermediate portion in the width direction Dw of the tire T while being brought into close contact with the maximum outer diameter portion 75 of the tire T and is brought into close contact with a tread part of the dent 73. The above-described shoulder portion S means a portion near the end portion in the width direction Dw in the tread part 70 coming into contact with the ground when a vehicle travels.

As shown in FIG. 3, the inner side probe 50S has sufficient rigidity that is not deformed when being pressed by the bead portion 71 and has electric conductivity. The inner side probe 50S in the embodiment is formed of a rod-shaped member. The inner side probe 50S is slightly inclined such that an end portion is disposed further toward an axial center side of the tire T than a base portion. With this, in a case where the width dimension of the tire T is shorter than the length dimension of the inner side probe 50S, or the like, the inner side probe 50S is not brought into contact with the bead portion 71 on a side in the width direction Dw opposite to the bead portion 71 to be measured.

A resistance measurement instrument (measurement unit) 60 is connected to the outer side probe 50A and the inner side probe 50S through wires W1 and W2.

The resistance measurement instrument 60 applies a predetermined measurement current, for example, between the outer side probe 50A and the inner side probe 50S, and measures a voltage across terminals in this case to measure electric resistance between the outer side probe 50A and the inner side probe 50S.

According to the above-described first embodiment, the outer side probe 50A extends in the width direction Dw of the tire T and is deformable following the radial direction Dr corresponding to the undulating shape of the tread part 70 in the width direction Dw. The conductive portion 54 is provided in at least the contact surface of the deformable portion 52 with the tread part 70 of the tire T and has electric conductivity. According to such a configuration, even though a part of the tire T in the width direction Dw is dented to the inside Dri in the radial direction Dr of the tire T, the deformable portion 52 and the conductive portion 54 can enter the dent 73 dented to the inside Dri in the radial direction Dr. For this reason, even in a case where a low electric resistance portion 100 of the tire T is positioned in the dent 73 dented to the inside Dri in the radial direction Dr of the tire T, it is possible to bring the conductive portion 54 into contact with the low electric resistance portion 100 to correctly measure the electric resistance of the tire T.

In the above-described first embodiment, in a case where the outer side probe 50A is brought into contact with the tread part 70 of the tire T, the deformable portion 52 enters the dent 73 dented to the inside Dri in the radial direction Dr of the tire T. For this reason, even in a case where the low electric resistance portion 100 is positioned in the dent 73 dented to the inside Dri in the radial direction Dr of the tire T, it is possible to bring the conductive portion 54 into contact with the low electric resistance portion 100.

In the above-described first embodiment, the electric resistance measurement device 1 and the outer side probe 50A further include the support member 51 having rigidity higher than the deformable portion 52. With this, when the deformable portion 52 is brought into contact with the tread part 70 of the tire T and is deformed in the radial direction Dr corresponding to the undulating shape of the tread part 70 in the width direction Dw, the support member 51 firmly supports the deformable portion 52 on the outside Dro in the radial direction Dr. With this, it is possible to make the deformable portion 52 stably enter the dent 73 more dented to the inside Dri in the radial direction Dr than the maximum outer diameter portion 75 of the tire T.

In the above-described first embodiment, the deformable portion 52 includes the conductive portion 54 and the elastically deformable body 53. In a case where the conductive portion 54 is brought into contact with the tread part 70 of the tire T, the conductive portion 54 is displaced to be pressed to the outside Dro in the radial direction Dr corresponding to the undulating shape of the tread part 70 of the tire T. The conductive portion 54 is pressed to the inside Dri in the radial direction Dr of the tire T by the elastically deformable body 53, and thus, enters the dent 73. For this reason, even in a case where the low electric resistance portion 100 is positioned in the dent 73 dented to the inside Dri in the radial direction Dr of the tire T, it is possible to bring the conductive portion 54 into contact with the low electric resistance portion 100.

In the above-described first embodiment, the conductive portion 54 is made of the band-shaped member 54t that extends in the width direction Dw and has flexibility and electric conductivity. With this, conductive portion 54 enters the dent 73 more dented to the inside Dri in the radial direction Dr than the maximum outer diameter portion 75 of the tire T. The band-shaped member 54t has electric conductivity, and thus, functions as the conductive portion 54. For this reason, even in a case where the low electric resistance portion 100 is positioned in a portion dented to the inside Dri in the radial direction Dr of the tire T, it is possible to bring the conductive portion 54 into contact with the low electric resistance portion 100.

In the above-described first embodiment, the elastically deformable body 53 is elastically deformed and compressed toward the outside Dro in the radial direction Dr, and exerts the pressing force P toward the inside Dri in the radial direction Dr with elasticity. With this, the conductive portion 54 is energized toward the inside Dri in the radial direction Dr of the tire T by the pressing force P. For this reason, it is possible to make the conductive portion 54 enter the dent 73 more dented to the inside Dri in the radial direction Dr than the maximum outer diameter portion 75 of the tire T.

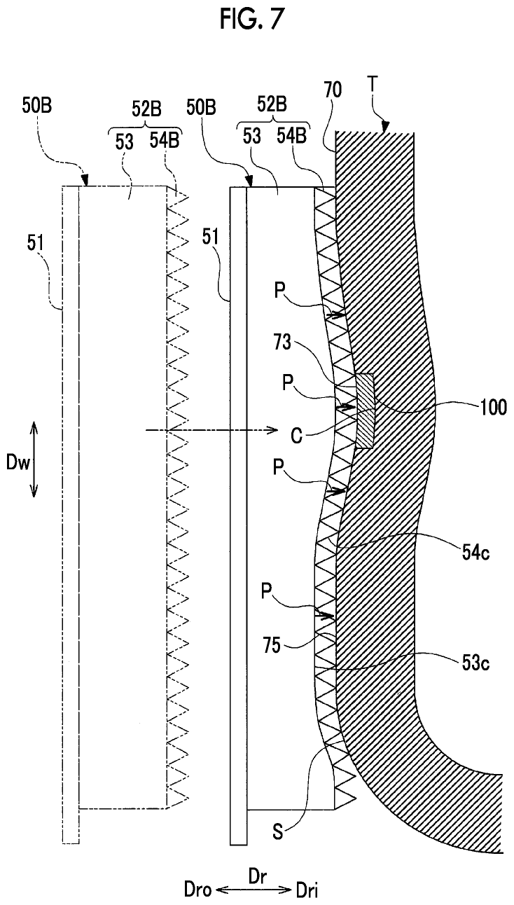

(Modification Example of First Embodiment) FIG. 7 is a sectional view showing a state in which an outer side probe of an electric resistance measurement device in a modification example of the embodiment is pressed to a tread part of a tire.

In the first embodiment, although the band-shaped member 54t is used as the conductive portion 54, the invention is not limited thereto.

As in the modification example of the first embodiment shown in FIG. 7, a coil spring 54c made of a material, such as metal, having electric conductivity may be used as a conductive portion 54B of an outer side probe (electric resistance probe) 50B. Similarly to the above-described band-shaped member 54t, the coil spring 54c is attached to the tip surface 53c of the elastically deformable body 53. In other words, the coil spring 54c is provided in the contact surface of the deformable portion 52B with the tread part 70.

With the above-described outer side probe 50B, similarly to the deformable portion 52 of the first embodiment, the deformable portion 52B relatively moves in the radial direction Dr of the tire T with respect to the tire T and is brought into contact in a range from the center portion C to the shoulder portion S the tread part 70 in the width direction Dw of the tire T.

More specifically, the coil spring 54c (conductive portion 54B) and the elastically deformable body 53 of the deformable portion 52B are pressed to the tread part 70 to be deformed along the undulating shape of the tread part 70 of the tire T in the width direction Dw. In this case, the elastically deformable body 53 is compressed and deformed toward the outside Dro in the radial direction Dr corresponding to the undulating shape of the tread part 70 of the tire T. The compressed and deformed elastically deformable body 53 energizes the coil spring 54c toward the inside Dri in the radial direction Dr by the pressing force P with elasticity. With this, the coil spring 54c enters the dent 73 formed in the intermediate portion in the width direction Dw of the tire T while being brought into contact with the maximum outer diameter portion 75 of the tire T and is brought into contact with the tread surface of the dent 73. Here, a portion in the coil spring 54c disposed on the inside Dri in the radial direction Dr is brought into contact with the tread part 70 of the tire T over the entire region in the width direction Dw of the tire T.

### Second Embodiment

Next, a second embodiment of the invention will be described referring to the drawings. The second embodiment is different from the first embodiment only in that an electric resistance probe is different. Accordingly, in the description of the second embodiment, the same portions as those in the first embodiment are represented by the same reference numerals while referring to FIG. 1 and overlapping description will not be repeated. That is, description of the overall configuration of the electric resistance measurement device 1 common to the configuration described in the first embodiment will not be repeated.

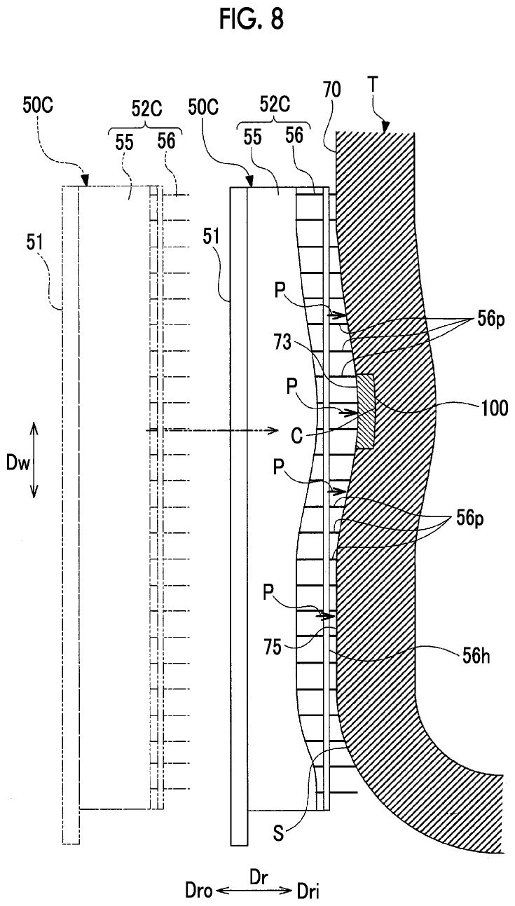

FIG. 8 is a sectional view showing a state in which an outer side probe of an electric resistance measurement device in the second embodiment is pressed to a tread part of a tire.

As shown in FIG. 1, the probe unit 6 of the electric resistance measurement device 1 in the second embodiment has an outer side probe (electric resistance probe) 50C and an inner side probe 50S.

As shown in FIG. 8, the outer side probe 50C includes a support member 51 and a deformable portion 52C.

The deformable portion 52C includes an elastically deformable body (pressing portion) 55 and a driven displaceable portion 56.

The driven displaceable portion 56 is provided at a position in the deformable portion 52C on the inside Dri in the radial direction Dr of the tire T. In other words, the driven displaceable portion 56 is provided at a position in the deformable portion 52C capable of being brought into contact with the tread part 70. The driven displaceable portion 56 includes a plurality of conductive pins (advance/retreat members) 56p and a holding member 56h.

A plurality of conductive pins (advance/retreat members) 56p are disposed at intervals in the width direction Dw of the tire T. A plurality of conductive pins 56p extend in the radial direction Dr of the tire T. Each conductive pin 56p can be formed of, for example, a material having electric conductivity, such as copper, silver, or aluminum.

The holding member 56h holds a plurality of conductive pins 56p in a state of being advanceable and retreatable in the radial direction Dr of the tire T. The holding member 56h shown in the second embodiment supports a plurality of conductive pins 56p to be slidable in the radial direction Dr. The holding member 56h has electric conductivity and is electrically connected to a plurality of conductive pins 56p. A plurality of conductive pins 56p described above are electrically connected to the resistance measurement instrument 60 (see FIG. 3) through the holding member 56h.

The holding member 56h is fixed to the support member 51. The holding member 56h extends in the width direction Dw in front view shown in FIG. 8. Similarly to the support member 51, the holding member 56h also has rigidity higher than the elastically deformable body 55. The rigidity of the holding member 56h may be equal to the rigidity of the support member 51.

The outer side probe 50C relatively moves in the radial direction Dr of the tire T with respect to the tire T, whereby each conductive pin 56p is brought into contact with the tread part 70 of the tire T. A plurality of conductive pins 56p are displaced to the outside Dro in the radial direction Dr corresponding to the undulating shape of the tread part 70 of the tire T. Specifically, the tip of each of a plurality of conductive pins 56p is pressed by the tread part 70 to be driven and is displaced corresponding to the undulating shape of the tread part 70 of the tire T.

Similarly to the elastically deformable body 53 in the first embodiment, the elastically deformable body 55 is supported by the support member 51. The elastically deformable body 55 can be formed of, for example, rubber or sponge.

A base end of each of a plurality of conductive pins 56p elastically deformable body 55. The elastically deformable body 55 is compressed and deformed (elastically deformed) toward the outside Dro in the radial direction Dr in a case where a plurality of conductive pins 56p are displaced in the radial direction Dr corresponding to the undulating shape of the tread part 70 of the tire T. The compressed and deformed elastically deformable body 55 presses a plurality of conductive pins 56p to the inside Dri in the radial direction Dr of the tire T by the pressing force P with elasticity.

With the above-described outer side probe 50C, the outer side probe 50C relatively moves the radial direction Dr of the tire T with respect to the tire T, whereby a plurality of conductive pins 56p of the driven displaceable portion 56 are brought into contact with the tread part 70 of the tire T. A plurality of conductive pins 56p are displaced to the outside Dro in the radial direction Dr along the undulating shape of the tread part 70 of the tire T in the width direction Dw, whereby the elastically deformable body 55 is deformed. Then, the elastically deformable body 55 exerts the pressing force P toward the inside Dri in the radial direction Dr and presses a plurality of conductive pins 56p toward the inside Dri in the radial direction Dr. With this, the driven displaceable portion 56 enters the dent 73 formed in the intermediate portion in the width direction Dw of the tire T while being brought into contact with the maximum outer diameter portion 75 of the tire T and is brought into contact with the tread surface of the dent 73. In this case, a portion (the tip of each of a plurality of conductive pins 56p) in the driven displaceable portion 56 disposed on the inside Dri in the radial direction Dr is brought into contact with the tread part 70 of the tire T over the entire region in the width direction Dw of the tire T.

According to the above-described second embodiment, the deformable portion 52C extends in the width direction Dw of the tire T and is deformable following the radial direction Dr corresponding to the undulating shape of the tread part 70 in the width direction Dw. The driven displaceable portion 56 is provided in at least the contact surface of the deformable portion 52C with the tread part 70 and has electric conductivity. With such a configuration, even in a case where the low electric resistance portion 100 is positioned in a portion dented to the inside Dri in the radial direction Dr of the tire T, it is possible to bring the driven displaceable portion 56 into contact with the low electric resistance portion 100 to correctly measure the electric resistance of the tire T.

In the above-described second embodiment, the driven displaceable portion 56 includes a plurality of conductive pins 56p that are provided at intervals in the width direction Dw and are provided advanceable and retreatable in the radial direction Dr. With such a configuration, the outer side probe 50C relatively moves in the radial direction Dr with respect to the tire T to be brought into contact with the tread part 70 of the tire T. With the contact with the tread part 70, each of the conductive pins 56p configuring the driven displaceable portion 56 is displaced to be pressed to the outside Dro in the radial direction Dr corresponding to the undulating shape of the tread part 70 of the tire T. A plurality of conductive pins 56p are pressed to the inside Dri in the radial direction Dr of the tire T by the elastically deformable body 55, and thus, enter the dent 73. For this reason, even in a case where the low electric resistance portion 100 is positioned in a portion dented to the inside Dri in the radial direction Dr of the tire T, it is possible to bring the conductive portion 54 into contact with the low electric resistance portion 100.

(Modification Example of Second Embodiment) In the second embodiment, although rubber, sponge, or the like is used as the elastically deformable body 55, the invention is not limited thereto. As the elastically deformable body 55, a spring member (not shown) that presses a plurality of conductive pins 56p individually to the inside Dri in the radial direction Dr of the tire T, such as a coil spring or a spring plate, may be used. The elastically deformable body 55 using such a spring member is compressed and deformed (elastically deformed) toward the outside Dro in the radial direction Dr corresponding to the undulating shape of the tread part 70 of the tire T. The compressed and deformed elastically deformable body 55 energizes a plurality of conductive pins 56p toward the inside Dri in the radial direction Dr with elasticity.

Instead of the elastically deformable body 55 of the second embodiment, an actuator (not shown) that presses a plurality of conductive pins 56p toward the inside Dri in the radial direction Dr of the tire T can be employed. In this case, a plurality of conductive pins 56p that are displaced to the outside Dro in the radial direction Dr corresponding to the undulating shape of the tread part 70 of the tire T may be pressed toward the inside Dri in the radial direction Dr by the actuator.

### Third Embodiment

Next, a third embodiment of the invention will be described referring to the drawings. The third embodiment is different from the second embodiment only in that an electric resistance probe is different. For this reason, in the description of the third embodiment, the same portions as those in the second embodiment are represented by the same reference numerals while referring to FIG. 1 and overlapping description will not be repeated. That is, description will be provided focusing on a difference from the second embodiment, and description of the configuration common to the configuration described in the first embodiment and the second embodiment will not be repeated.

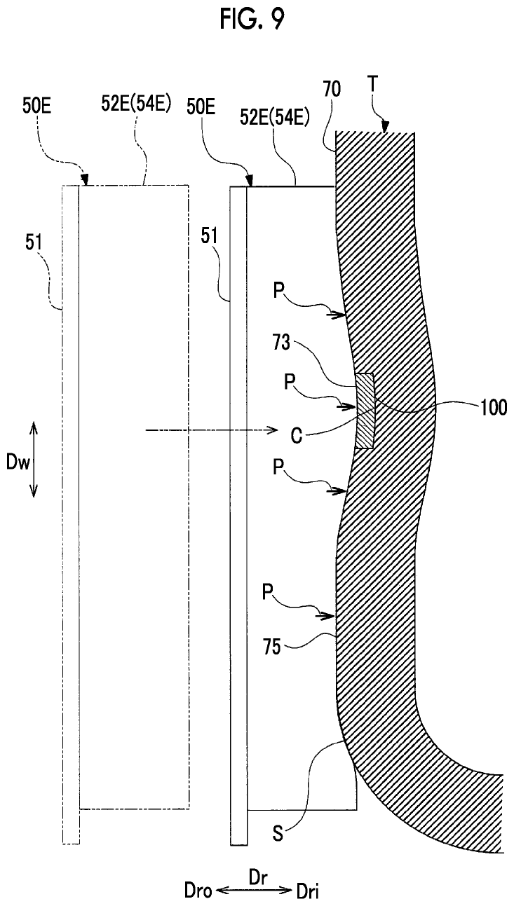

FIG. 9 is a sectional view showing a state in which an outer side probe of an electric resistance measurement device in the third embodiment is pressed to a tread part of a tire.

As shown in FIG. 1, the probe unit 6 of the electric resistance measurement device 1 of the tire T has an outer side probe (electric resistance probe) 50E and an inner side probe 50S.

As shown in FIG. 9, the outer side probe 50E includes a support member 51 and a deformable portion 52E.

Similarly to the elastically deformable body 53 in the above-described first embodiment, the deformable portion 52E is supported by the support member 51. The deformable portion 52E extends in the width direction Dw of the tire T and is deformable in the radial direction Dr corresponding to the undulating shape of the tread part 70 in the width direction Dw. The deformable portion 52E is formed of, for example, rubber or sponge. The deformable portion 52E is compressed and deformed toward the outside Dro in the radial direction Dr corresponding to the undulating shape of the tread part 70 of the tire T in a case of relatively moving in the radial direction Dr of the tire T with respect to the tire T to be brought into contact with the tread part 70 of the tire T. The compressed and deformed deformable portion 52E exerts the pressing force P toward the inside Dri in the radial direction Dr with elasticity. The deformable portion 52E has electric conductivity by kneading particles made of metal, carbon black, or the like having electric conductivity. That is, the deformable portion 52E is also used as a conductive portion 54E as a whole. The conductive portion 54E is electrically connected to the resistance measurement instrument 60 (see FIG. 3).

According to the above-described third embodiment, the deformable portion 52E of the outer side probe 50E extends in the width direction Dw of the tire T and is deformable in the radial direction Dr corresponding to the undulating shape of the tread part 70 in the width direction Dw. With this, the deformable portion 52E can enter the dent 73 dented to the inside Dri in the radial direction Dr. For this reason, even in a case where the low electric resistance portion 100 is positioned in a portion dented to the inside Dri in the radial direction Dr of the tire T, it is possible to bring the deformable portion 52E (conductive portion 54E) into contact with the low electric resistance portion 100 to correctly measure the electric resistance of the tire T. Furthermore, since the portion of the deformable portion 52E that is elastically deformed is also used as the conductive portion 54E, it is possible to efficiently perform manufacturing or the like of the outer side probe 50E.

### Other Embodiments

The invention is not limited to the above-described embodiments, and design changes can be made without departing from the spirit and scope of the invention.

For example, in each embodiment and each modification example described above, the upper end portion of the outer side probe 50A, 50B, 50C, or 50E is disposed at a slightly higher position in the height direction than the center portion C of the tire T. However, the height of the upper end portion of the outer side probe 50A, 50B, 50C, or 50E is not limited to the above height. For example, the upper end portion of the outer side probe 50A, 50B, 50C, or 50E may be disposed at a height position equal to or higher than the center portion C at the highest position among the center portions C of a plurality of types of tires T assumed as a target to be inspected.

In each embodiment and each modification example described above, an example where the two outer side probes 50A, 50B, 50C, or 50E are disposed in parallel in the circumferential direction has been described. However, only one outer side probe 50A, 50B, 50C, or 50E may be disposed. In each embodiment and each modification example described above, a case where only one inner side probe 50S is disposed has been described. However, a plurality of inner side probes 50S may be provided in the circumferential direction.

In each embodiment and each modification example described above, although a case where the inner side probe 50S is disposed inclined has been described, the inner side probe 50S may be disposed to extend vertically upward or an inclination angle may be changeable as needed.

In the above-described embodiments, although a case where the probe unit 6 is displaced in the up-down direction by the lifting/lowering mechanism 12 has been described, the direction of displacing the probe unit 6 is not limited to the up-down direction, and may be a direction corresponding to the posture of the tire T at the time of transfer.

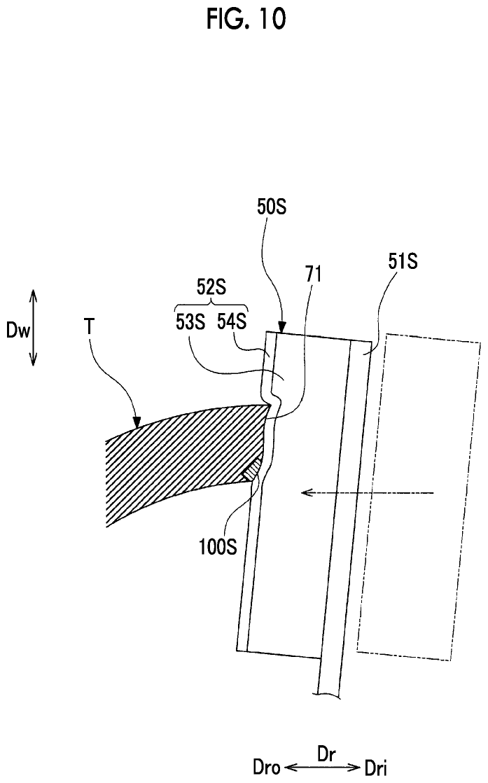

FIG. 10 is a diagram showing an inner side probe in a modification example of the embodiment of the invention.

In the above-described embodiments, a case where only the outer side probe 50A, 50B, 50C, or 50E among the inner side probe 50S and the outer side probe 50A, 50B, 50C, or 50E is deformable following the radial direction Dr corresponding to the undulating shape of the tire T has been described.

However, like the inner side probe 50S in the modification example shown in FIG. 10, the inner side probe 50S may have the same configuration as the above-described outer side probe 50A, 50B, 50C, or 50E, that is, may be configured to be deformable following the radial direction Dr corresponding to the undulating shape of the tire T.

As shown in FIG. 10, the inner side probe 50S in the modification example relatively moves to the outside Dro in the radial direction Dr with respect to the tire T to be brought into contact with the bead portion 71 formed in the inner peripheral portion of the tire T. The inner side probe 50S includes a support member 51S and a deformable portion 52S. The deformable portion 52S includes an elastically deformable body 53S and a conductive portion 54S. The support member 51S is configured similarly to any one of the support members 51 in the above-described embodiments. The deformable portion 52S is configured similarly to any one of the deformable portions 52, 52B, 52C, and 52E in the above-described embodiments.

The inner side probe 50S in the modification example of the embodiment in this way is deformable following the radial direction Dr corresponding to the undulating shape of the bead portion 71 and has electric conductivity in at least the contact surface with the bead portion 71. For this reason, it is possible to stably bring the conductive portion 54S of the inner side probe 50S into contact with an electric conduction portion 100S exposed in the bead portion 71.

### INDUSTRIAL APPLICABILITY

With the tire electric resistance measurement device and the electric resistance probe described above, it is possible to improve reliability in electric resistance measurement of a tire.

### REFERENCE SIGNS LIST

1: electric resistance measurement device
2: roller conveyor
3: roller
4: side wall
6: probe unit
8: floor
9: stand
10: leg portion
11: beam
12: lifting/lowering mechanism
13: base portion
14: upper support plate
15: lower support plate
16: guide rod
17: guide portion
18: guide tube
19: frame portion
20: support arm
21: fluid pressure cylinder
22: outer tube
23: inner rod
29: base plate
30: guide rod
31: frame body
32: first slide portion
33: second slide portion
34: fluid pressure cylinder for probe
35: inner rod
36: outer tube
42: first support metal fitting
47: second support metal fitting
50A, 50B, 50C, 50E: outer side probe (electric resistance probe)
50S: inner side probe
51: support member
51a: base portion
51b: side wall portion
52, 52B, 52C, 52E: deformable portion
52k: screw
53, 55: elastically deformable body (pressing portion)
53a: base surface
53b: side surface
53c: tip surface
54, 54B: conductive portion (driven displaceable portion)
54E: conductive portion
54c: coil spring
54t: band-shaped member
56: driven displaceable portion
56h: holding member
56p: conductive pin (advance/retreat member)
60: resistance measurement instrument
70: tread part
71: bead portion
73: dent
75: maximum outer diameter portion
100: low electric resistance portion
C: center portion
Dr: radial direction
Dri: inside
Dro: outside
Dw: width direction
P: pressing force
S: shoulder portion
T: tire
W1: wire
W2: wire
i: insulating member

1. A tire electric resistance measurement device comprising:
an inner side probe that is disposed on an inner periphery side of a tire and is capable of being brought into contact with an inner peripheral portion of the tire; and
an outer side probe that is disposed on an outer periphery side of the tire and is capable of being brought into contact with a tread part of the tire by relatively moving in a radial direction of the tire with respect to the tire,
wherein the outer side probe extends in a width direction of the tire, is deformable following the radial direction corresponding to an undulating shape of the tread part in the width direction, and has electric conductivity in at least a contact surface with the tread part.

2. The tire electric resistance measurement device according to claim 1,
wherein the outer side probe enters a dent more dented to an inside in the radial direction than a maximum outer diameter portion of the tire in an intermediate portion of the tire in the width direction in a case where the outer side probe is brought into contact with the tread part of the tire by relatively moving in the radial direction of the tire with respect to the tire.

3. The tire electric resistance measurement device according to claim 1, further comprising:
a support member that has rigidity higher than the outer side probe, extends in the width direction outside the tire in the radial direction with respect to the outer side probe, and supports the outer side probe.

4. The tire electric resistance measurement device according to claim 1,
wherein the outer side probe includes a driven displaceable portion that is displaced to an outside in the radial direction corresponding to the undulating shape of the tread part of the tire in a case where the driven displaceable portion is brought into contact with the tread part of the tire by relatively moving in the radial direction of the tire with respect to the tire, and a pressing portion that presses the driven displaceable portion to an inside in the radial direction of the tire.

5. The tire electric resistance measurement device according to claim 4,
wherein the driven displaceable portion is a band-shaped member that extends in the width direction and has flexibility and electric conductivity.

6. The tire electric resistance measurement device according to claim 4,
wherein the driven displaceable portion is a plurality of advance/retreat members that are provided at intervals in the width direction and are provided advanceable and retreatable in the radial direction.

7. The tire electric resistance measurement device according to claim 4,
wherein the pressing portion is formed to be compressible by being elastically deformed toward the outside in the radial direction corresponding to the undulating shape of the tread part of the tire in a case where the pressing portion is brought into contact with the tread part of the tire by relatively moving in the radial direction of the tire with respect to the tire.

8. The tire electric resistance measurement device according to claim 1,
wherein the outer side probe is elastically deformable toward an outside in the radial direction corresponding to the undulating shape of the tread part of the tire in a case where the outer side probe is brought into contact with the tread part of the tire by relatively moving in the radial direction of the tire with respect to the tire, and has electric conductivity.

9. An electric resistance probe that extends in a width direction of a tire, is deformable following a radial direction of the tire corresponding to an undulating shape of the tire in the width direction in a case where the electric resistance probe is brought into contact with the tire by relatively moving in the radial direction of the tire with respect to the tire, and has electric conductivity in at least a contact surface with the tire.
